(19) United States
(12) Patent Application Publication
(10) Pub. No.: US 2026/0355057 A1
(43) Pub. Date: Nov. 12, 2026

(54) MEDICAMENT CAPSULE HOLDER FOR SYRINGE

(71) Applicants: Yuval ERAN, Shoham (IL); Adar GAT, Shoham (IL)

(72) Inventors: Yuval ERAN, Shoham (IL); Adar GAT, Shoham (IL)

(21) Appl. No.: 19/482,226

(22) PCT Filed: May 7, 2024
(86) PCT No.: PCT/IL2024/050434

§ 371 Date: Nov. 6, 2025

(30) Foreign Application Priority Data
May 7, 2023 (IL) 302735

(51) Int. Cl. A61J 1/16; A61J 1/06; A61M 5/178

(52) U.S. Cl. CPC A61J 1/16; A61J 1/065; A61M 5/1782; A61M 2209/045

(57) ABSTRACT

An ampoule mate for retaining a medicament ampoule in communication with a syringe, comprising a main body with a throughgoing bore having a syringe lock on one side and an ampoule lock on another side. The bore has, at one end, a narrowing portion that ends in an ampoule neck lock. In use, the medicament ampoule is inserted into the bore until the neck of the ampoule rests against the neck lock; the ampoule can then be locked in place by the ampoule lock. The neck lock is configured so that, when sideward pressure is applied to the ampoule's neck above the neck lock, the neck breaks, leaving a smooth and even edge to the neck. After the medicament is drawn into the syringe, the syringe can be firmly held to the ampoule mate by the syringe lock.

### FIELD OF THE INVENTION

The present invention generally pertains to a system and method for holding a medicament capsule in communication with a syringe.

### BACKGROUND OF THE INVENTION

Life-saving medical treatment often involves intravenous (IV), intramuscular (IM), Intraosseous (IO) or intranasal (IN) medications. Preparing a syringe for injection of IV, IM, IO or IN medications involves a series of steps, such as taking an ampoule of medication, breaking it open, inserting a syringe into the opened ampoule and drawing the medication into the syringe, all of which can be done in advance, so that a prepared syringe is available when needed.

However, in such a multi-step process, mistakes can be, and all too often, are made. For example, errors occur in at least 10.1% of IV injections the UK (Incidence and prevalence of intravenous medication errors in the UK: a systematic review, 2018), and in 23% of IV injection in Germany (Prevalence of intravenous medication administration errors: a cross-sectional study, 2017). In 32.1% of cases in the UK, the wrong medication is used. 60% of life-threatening incidents involve IV drugs.

In the USA, errors in the administration of IV, IM, IO or IN medications is the eighth largest cause of death. It is estimated that costs in the USA are estimated to be 77 billion dollars per year (https://www.amcp.org/about/managed-care-pharmacy-101/concepts-managed-care-pharmacy/medication-errors).

The most common reasons for error are:
1. Working long hours leading to mistakes due to tiredness.
2. Not verifying the drug before administrating.
3. Not marking the medication on the syringe. In one study, it was found that, when administering medication, 1 in 4 nurses prepare the syringes in advance but never label them.
4. Administering the medication to the wrong patient.
5. Not knowing the correct medication.
6. Not providing the correct medication.

Many of these causes of error can be prevented by providing appropriate protocols and ensuring that medical personnel have the appropriate knowledge and skills.

However, protocols and education can only do so much, especially when people are tired or rushed. WIPO Patent Application Publication No. WO2011/116462 teaches a syringe and vial, where the vial comprises an identifier that indicates the substance that is within the vial. The vial and syringe are configured such that when the syringe is used to draw the substance from the vial, the identifier is transferrable from the vial to the syringe so that the identifier is then secured to the syringe. Accordingly, the syringe, when loaded with the substance from the vial, comprises the identifier, which now indicates the substance that is within the syringe.

However, in the system of WO2011/116462, the identifier is part of or is attached to a cap on the vial of medicament. After filling of the syringe, when the syringe is removed from the vial, the cap of the vial is transferred to a fitting near the tip of the syringe and is retained there by friction. Therefore, pressure on the cap that rotates it relative to the syringe can cause disengagement between the cap and the syringe, thereby separating the identifier from the syringe. Furthermore, the system requires both a proprietary syringe and a proprietary vial, severely restricting the number of medications that could be used.

U.S. Pat. No. 9,901,682 teaches a medical syringe apparatus including a syringe unit, a hypodermic needle attached to the syringe unit and at least one interfacing member for mating a labeled medication container to the syringe unit. A filling needle is attached to the syringe unit for drawing fluid from a container. A movable needle sheath protects the hypodermic needle when not in use and may be moved out of the way when administering an injection. The medication label is visible. The container remains mated to the syringe when the syringe is in use. Risk of mislabeling the syringe may therefore be substantially reduced or eliminated and patient safety may accordingly be improved. The vial may be coupled to the syringe by a tether or by clips, with the tether or clips being integral to the syringe or added to the syringe during manufacture.

However, U.S. Pat. No. 9,901,682 teaches a device requiring a proprietary syringe, with flexible clip mechanisms that would allow separation of the ampoule from the syringe.

EP patent application publication no. EP2525766A1 teaches a device having a portion configured for snapping off a snap-top of the ampoule. This comprises a collar, in particular sized to fit the snap-top of an ampoule (ampoules are of standard, albeit different, sizes). In embodiments of this latter arrangement, the syringe fitting portion clips to the syringe and the ampoule-mounting portion clips to the ampoule in embodiments by a collar around the neck of the snap-top. In embodiments the collar is hingedly attached to the syringe-fitting portion (and may be folded back onto the syringe fitting portion after use). This enables the syringe-fitting portion to be articulated with respect to the ampoule-mounting portion (collar) to snap the top off the ampoule, which may then be removed from the device and filled in the normal way. In this approach the device remains attached to the syringe, along with the label it bears. In such an approach preferably the device is shipped together with the syringe and ampoule so that the label, which is attached to the device, remains on the syringe after aspiration of the drug from the ampoule.

However, in some embodiments of EP2525766, a label must be manually removed from the device and attached to the syringe. In other embodiments, the device is provided with a labelled collar and, in use, the collar is automatically transferred from the device to the distal (needle-end) of the syringe.

In either type of embodiment, the ampoule is a proprietary item, as it must be provided along with an appropriate label.

It is therefore a long felt need to provide a method of preventing the identity of the medication to be administered for being separated from the syringe used to administer the medication that does not require manual labeling, does not require a proprietary ampoule, a proprietary syringe, or both a proprietary syringe and ampoule, and does not require a different sized device for different sizes of ampoules or syringes.

### SUMMARY OF THE INVENTION

It is an object of the present invention to disclose a system for holding a medicament capsule in communication with a syringe.

It is another object of the present invention to disclose an ampoule mate for retaining a medicament ampoule in communication with a syringe, comprising:
a main body (110) with a throughgoing cylindrical bore (155), the bore (155) being parallel to a main longitudinal axis of the main body (110);
a syringe lock (120) extending from a side of the main body (110), the syringe lock (120) configured to firmly hold a syringe; the side of the main body between the arms of the syringe lock (120) having a concave curve, the concave curve having a longitudinal axis parallel to the main longitudinal axis of the main body (110); and
an ampoule lock (170) extending from a side of the main body (110) outside the syringe lock (120), the ampoule lock (170) configured to firmly hold a medicament ampoule (200).

It is another object of the present invention to disclose the ampoule mate as described in any of the above, wherein the main body further comprises an ampoule neck lock (130) at a distal end of the bore configured to assist in ensuring, in use, that a neck of a medicament ampoule (200) breaks evenly when a sidewards pressure is applied to the neck of the medicament ampoule (200).

It is another object of the present invention to disclose the ampoule mate as described in any of the above, wherein said syringe lock is configured to accept a syringe of a size between 1 cc and 10 cc. It is another object of the present invention to disclose the ampoule mate as described in any of the above, wherein said ampoule lock is configured to accept an ampoule of a size between 1 cc and 10 cc.

It is another object of the present invention to disclose the ampoule mate as described in any of the above, wherein said syringe lock (120) is configured as a cable tie.

It is another object of the present invention to disclose the ampoule mate as described in any of the above, wherein said main body (110) further comprises a pen hanger (180).

It is another object of the present invention to disclose the ampoule mate as described in any of the above, wherein said ampoule lock (170) comprises a pin (173) configured to secure said ampoule, said pin snugly fittable into a hole (177) in a side of said main body (110), said hole (177) extending from an outside of said main body (110) to said bore (155), an end of said pin (173) extending into said bore (155), said pin (173) configured to be movable inwards towards said bore (155) but not movable outwards from said bore (155).

It is another object of the present invention to disclose the ampoule mate as described in any of the above, additionally comprising a needle ledge guide (160) at a distal end of said main body (110) for protecting the fingers of the operator during controlled breakage of said ampoule.

It is another object of the present invention to disclose a method of preparing a syringe for use, comprising steps of:
providing a syringe with a needle;
providing an ampoule of medication;
providing an ampoule mate comprising:
   a main body (110) with a throughgoing cylindrical bore (155), the bore (155) being parallel to a main longitudinal axis of the main body (110);
   a syringe lock (120) extending from a side of the main body (110), the syringe lock (120) configured to firmly hold a syringe; the side of the main body between the arms of the syringe lock (120) having a concave curve, the concave curve having a longitudinal axis parallel to the main longitudinal axis of the main body (110); and
   an ampoule lock (170) extending from a side of the main body (110) outside the syringe lock (120), the ampoule lock (170) configured to firmly hold a medicament ampoule; (200).
inserting the ampoule (200) into the bore (155) of the ampoule mate (100);
rotating the ampoule relative to the ampoule mate to break the neck of the ampoule;
inserting the needle into the ampoule (200) and drawing the contents of the ampoule (200) into the syringe (300);
placing the side of the syringe against the curved side of the ampoule mate; and
closing the syringe lock (120), thereby attaching the syringe (300) to the ampoule mate (100).

It is another object of the present invention to disclose the method as described in any of the above, additionally comprising a step of prividing the main body further comprising an ampoule neck lock (130) at a distal end of the bore (155) configured to assist in ensuring, in use, that a neck of a medicament ampoule (200) breaks evenly when a sidewards pressure is applied to the neck of the medicament ampoule;

It is another object of the present invention to disclose the method as described in any of the above, additionally comprising a step of configuring said syringe lock to accept a syringe of a size selected from the group consisting of 2.5 cc, 3 cc, 5 cc and 10 cc.

It is another object of the present invention to disclose the method as described in any of the above, additionally comprising a step of configuring said ampoule lock to accept an ampoule of a size between 1 cc and 10 cc.

It is another object of the present invention to disclose the method as described in any of the above, additionally comprising a step of configuring said syringe lock (120) as a cable tie.

It is another object of the present invention to disclose the method as described in any of the above, additionally comprising a step of providing said main body (110) comprising a pen hanger (180). It is another object of the present invention to disclose the method as described in any of the above, additionally comprising a step of providing said ampoule lock (170) comprising a pin (173) configured to secure said ampoule, said pin snugly fittable into a hole (177) in a side of said main body (110), said hole (177) extending from an outside of said main body (110) to said bore (155), an end of said pin (173) extending into said bore (155), said pin (173) configured to be movable inwards towards said bore (155) but not movable outwards from said bore (155).

It is another object of the present invention to disclose the method as described in any of the above, additionally comprising a step of providing said main body with a needle ledge guide (160) at a distal end of said main body (110) for protecting the fingers of the operator during controlled breakage of said ampoule.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1A-D depicts embodiments of the prior art;

FIG. 2 schematically illustrates an embodiment of the ampoule mate;

FIG. 3 illustrates another embodiment of the capsule lock mechanism;

FIG. 4A-B illustrates a perspective view another embodiment of the ampoule mate;

FIG. 5A-C illustrates a bottom view, a top view and a plan view of another embodiment of the ampoule mate;

FIG. 6A-C schematically illustrate a method of use of the ampoule mate; and

FIG. 7 illustrates an embodiment of the system as ready to use, with the syringe and ampoule held firmly by the ampoule mate; and

FIG. 8A-B, 9A-F, 10A-B, 11, 12, 13, 14, 15, 16, 17, 18, 19, illustrate embodiments of the ampoule mate system.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for holding a medicament capsule in communication with a syringe.

The term ‘ampoule’ hereinafter refers to a container, typically of glass, configured to hold a predetermined quantity of a medicament of a type deliverable via a syringe. An ampoule is typically sealed during storage of the medicament. The term ampoule, as used herein, will also refer to any container for a medication for injection of a size small enough to be held by any embodiment of an ampoule mate as disclosed herein. Non-limiting terms for such a container comprise an ampoule, a vial, a phial or a flacon.

The term ‘ampoule mate system’ hereinafter refers to the ampoule mate in its ready-to-use configuration, comprising an ampoule mate holding an ampoule and holding a syringe containing the medicament drawn from the ampoule into the syringe.

Life-saving medical treatment often involves intravenous (IV), intramuscular (IM), Intraosseous (IO) or intranasal (IN) medications. Preparing a syringe for injection of IV, IM, IO or IN medications involves a series of steps, such as taking an ampoule of medication, breaking it open, inserting a syringe into the opened ampoule and drawing the medication into the syringe, all of which can be done in advance, so that a prepared syringe is available when needed.

However, in such a multi-step process, mistakes can be and, all too often, are made.

In order to help prevent such errors, the device of the present invention has been developed, to ensure that the ampoule that contained the medication remains in communication with the syringe administering the medication so that the label on the ampoule can be read to check that the correct medication is administered.

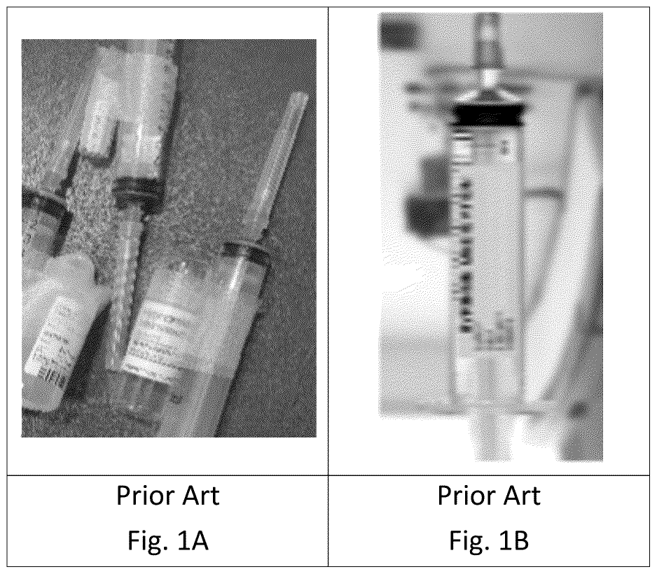

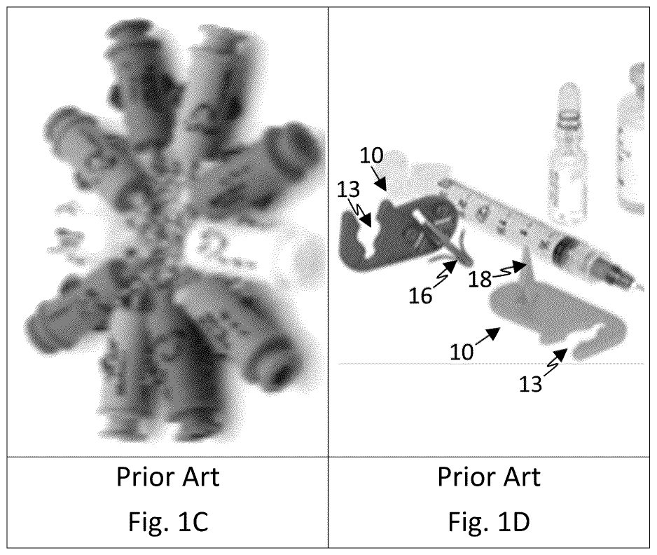

Attempts have been made in the past to ensure that the medication in a syringe is properly identified. Some of the prior art is shown in FIG. 1A-D.

As shown in in FIG. 1A, the ampoule can be taped to the syringe. However, this can make it awkward to read the label on the ampoule, especially if the tape is not fully transparent, and it can be awkward to hold both the syringe and the ampoule in one hand while applying the tape with the other, all the while making sure that the ampoule label is facing outwards so as to remain legible.

FIG. 1B shows a label attachable to a syringe. This would require a stock of labels of different sizes, and for the medical personnel to copy the information from the ampoule onto the syringe, remove the label from backing paper and stick it onto the syringe, a time-consuming and somewhat awkward procedure that is often overlooked; in some cases, one out of four nurses never put labels on the syringes they prepare.

FIG. 1C shows ampoules with loops on the side, through which a string or cable tie can be threaded, by means of which the ampoule can be held against the syringe. However, this requires use of the proprietary ampoule and further requires threading the string or cable tie through the loops before tying the string to the syringe or tightening the cable tie against the syringe. In addition, a loop can break during the threading or tightening process, making the connection to the syringe insecure.

FIG. 1D shows a device (10) clippable to a syringe by a clip (13), with spikes (16, 18) that can be inserted in an ampoule to prevent the ampoule from getting separated from the syringe. However, the device (10) significantly increases the footprint of a syringe plus ampoule. In addition, different sizes of device (10) would be needed for different size syringes, and different types of spike (16, 18) would be needed for different sizes and shapes of ampoule.

The ampoule mate of the present invention is compatible with ISO 9187 (Ampoules) and ISO 7886 (Syringes). Under the CE, it is a class 1 device under rule 1, in that it is low risk, is provided non-sterile and does not have a measuring function. Similarly, under the FDA, it is a rule 1 device, presenting minimal potential for harm.

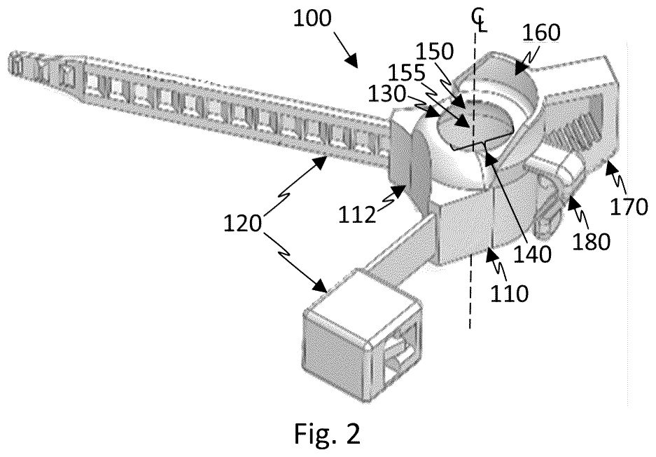

FIG. 2 illustrates an embodiment of the ampoule mate (100) of the present invention. The ampoule mate (100) comprises a main body (110) with a syringe lock (120), an ampoule holder (150), the ampoule holder (150) being a throughgoing bore in the main body (110), an ampoule lock (170) and a pen hanger (180). The closed end of the pen hanger (180) is near the proximal end of the ampoule mate (100), the end that, in use, will face toward the syringe's plunger flange. In use, the distal end of the ampoule mate (100) faces toward the syringe needle hub and the needle.

In some embodiments of the present invention, the shape of the pen hanger (180) may vary, but the pen hanger (180) is configured to removably hold the ampoule mate system in place on a pocket.

In use, a syringe (not shown) is held between a curved side (112) of the main body (100) and a syringe lock (120). The longitudinal axis of the curved side is parallel to the main longitudinal axis (CL) of the main body (110).

The syringe lock (120) comprises a cable tie so that it is adjustable and can hold syringes of the most common sizes, between 1 cc and 10 cc, for example, 2.5 cc, 3 cc, 5 cc and 10 cc syringes. A cable tie being a one-way mechanism, the syringe will remain attached to the ampoule mate (100) after the cable tie is pulled tight.

It should be noted that, if desired, the cable tie can be clipped short after attachment to the syringe, to reduce the in-use footprint of the ampoule mate system, namely an ampoule mate holding a syringe and an ampoule, and to prevent tangling of ampoule mate systems while they are stored in a container.

The ampoule holder (150) comprises a bore in the main body (110) sized to accept common sizes of ampoule between 1 cc and 10 cc. The bore is cylindrical, with its longitudinal axis being parallel to the main longitudinal axis of the main body (110). In some embodiments, the ampoule holder (150) is configured to accept common sizes of ampoule up to 25 cc. The minimum ampoule size can be in a range from 1 cc to 25 cc, and the maximum ampoule size can be in a range from 1 cc to 25 cc.

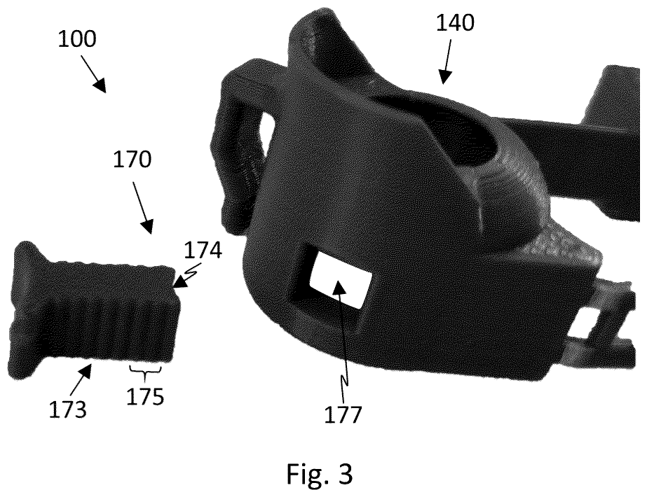

The ampoule lock (170) is configured to hold the ampoule firmly in place. An embodiment of an ampoule lock is shown in FIG. 2; with another embodiment of the ampoule lock (170) shown, disassembled, in FIG. 3. An ampoule lock (170) can comprise any conventional non-return locking mechanism of a size and shape configured to securely hold an ampoule of a size between 1 cc and 10 cc, for non-limiting example, a second cable tie or a snap-fit mechanism.

In the embodiment of FIG. 2, the ampoule lock (170) comprises an arm (173) attached to the main body (110), the distal end of the arm (173) comprising a series of teeth (175) that match with teeth (not shown) in a hole (not shown), forming a non-return locking mechanism so that, when the arm (173) is in the hole (177), it can be pushed in to hold the ampoule but it can not be pulled out, so the ampoule will be firmly held by the ampoule mate (100).

In the embodiment of FIG. 3, the ampoule lock (170) comprises a pin (173) and a matching hole (177) in the main body (110). The pin (173) comprises a series of teeth (175) that match with teeth (not shown) in the hole (177), forming a non-return locking mechanism so that, when the pin (173) is in the hole (177), it can be pushed in to hold the ampoule but it can not be pulled out, so the ampoule will be firmly held by the ampoule mate (100).

The ampoule mate (100) further comprises an ampoule neck lock (130) at a distal end of the ampoule holder (150) to assist in breaking the ampoule safely by applying isometric pressure to the ampoule neck and a needle ledge guide (160) at the distal end of the main body (110). The needle ledge guide (160) is configured to protect the fingers of the operator during controlled breakage of the ampoule and to assist in withdrawing safely the contents of the ampoule into the syringe by guiding the syringe as the needle or the syringe tip enters the ampoule and by supporting the distal end of the syringe as the contents of the ampoule are withdrawn into the syringe.

In addition, the main body (110) can comprise a pen hanger (180) to enable the ampoule mate to be clipped to a pocket to enable medical personnel to carry the filled syringe (and attached ampoule) safely, conformably and conveniently.

The syringe mate (100) can be attached to a belt or a belt loop by means of the pen hanger (180). In some embodiments, instead of or in addition to the pen hanger (180), the syringe mate (100) can comprise an additional part (not shown), a hanger (not shown) or an add-on (not shown) configured to allow the ampoule mate system to be easily attached to or easily carrier from a belt. In preferred embodiments, the ampoule mate (100) is made of transparent material. This allows the text on the ampoule, especially the medicament description, to be read, no matter what the orientation of the ampoule in the ampoule mate.

In some embodiments, the ampoule mate (100) comprises a lens (not shown) to enlarge the text on the ampoule and make it easier to read. Preferably, the lens comprises a portion of the side of the ampoule mate (100), although it can comprise an attachment to the ampoule mate (100). The lens can comprise a convex lens, a biconvex lens, a Fresnel lens, or any conventional system configured to readably enlarge text.

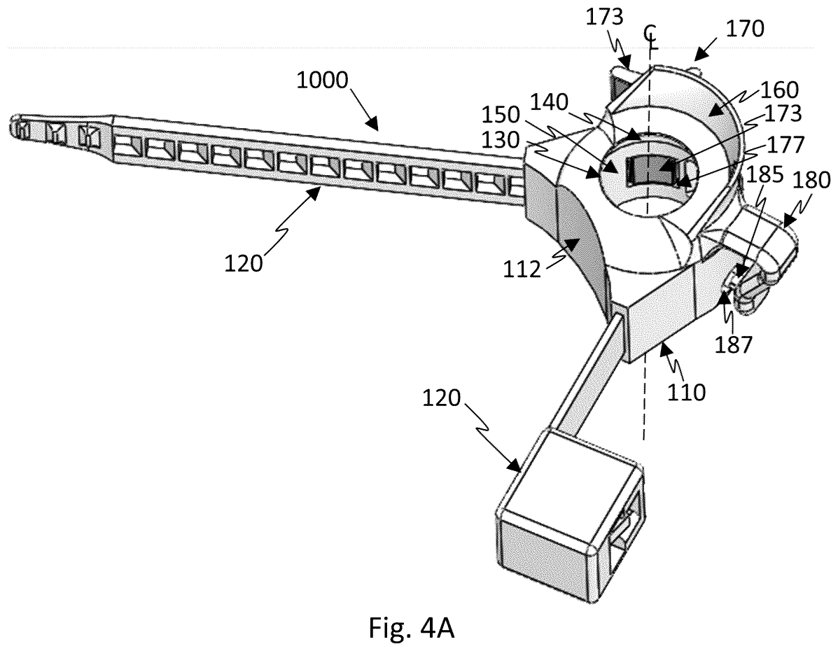

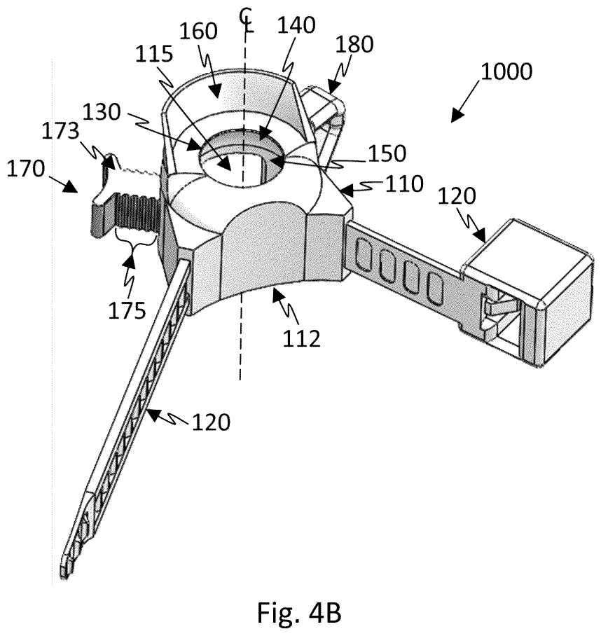

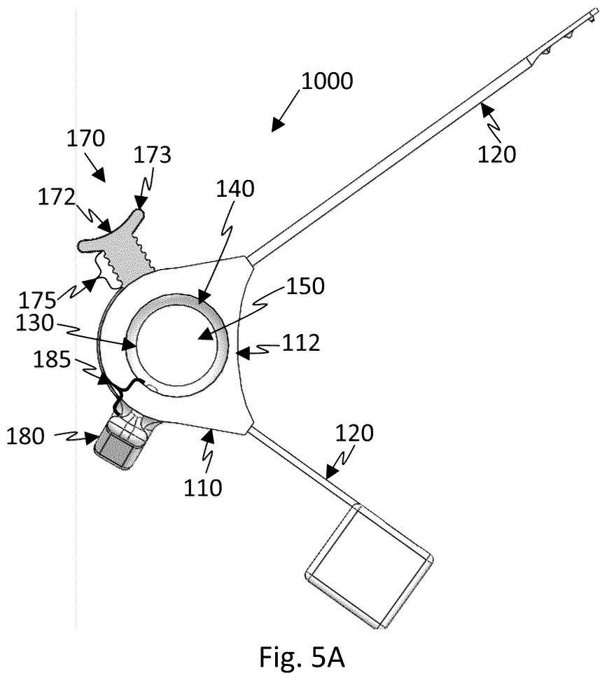

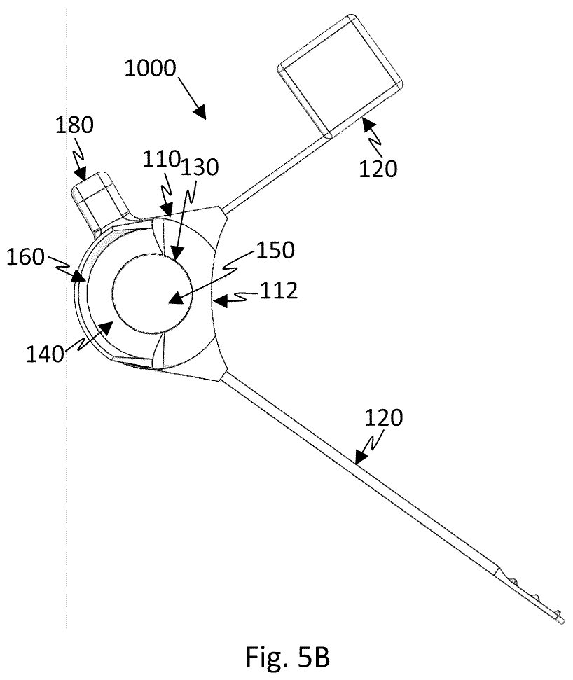

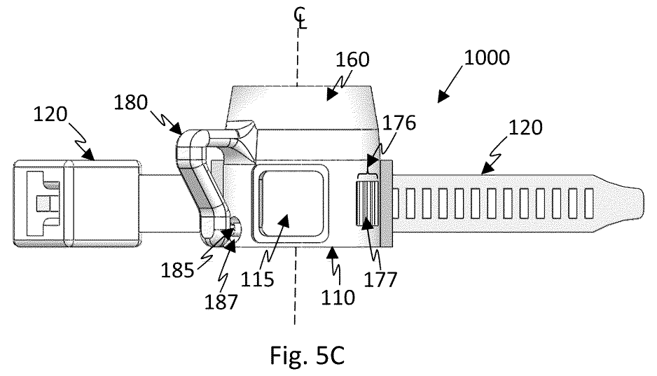

It is known that up to 5% of the contents of an ampoule may remain in the ampoule after the syringe is withdrawn. In some embodiments, the ampoule mate (100) comprises a part (not shown, see FIGS. 4-5, below) configured to assist in removal of residual liquid from the ampoule by encouraging the residual liquid to flow down the sides of the ampoule and collect in the base of the ampoule so that the residual liquid can be sucked up into the syringe. Embodiments of the part can comprise, for non-limiting example, a tapper or shaker configured, when activated, to strike the ampoule so as to mechanically simulate the tapping of the ampoule well-known in the art. A non-limiting example of a tapper comprises a spring-loaded weight. When the spring is released from a compressed configuration or pulled into an extended configuration and released, the weight swings repeatedly into contact with a side of the ampoule. The release or extension-and-release mechanism can comprise a button, lever or switch activator; spring-loaded mechanisms and activators for them are well-known in the art.

FIG. 4A-B and 5A-C illustrate another embodiment of the ampoule mate (1000). FIG. 4A-B shows perspective views of the modified ampoule mate (1000), while FIG. 5A-C show, respectively, a bottom view (FIG. 5A), a top view (FIG. 5B)and a plan view (FIG. 5C) of the ampoule mate (1000). The modified ampoule mate (1000) is similar to the first embodiment of the ampoule mate (100), as shown in FIGS. 2-3 in that it comprises a syringe lock (120), a body (110) comprising an ampoule holder (150) with an ampoule retainer (140) at one end of the ampoule holder (150). The ampoule holder (150) and ampoule retainer (140) form a throughgoing bore along the longitudinal axis of the main body. The bore terminates in an ampoule neck lock (130), a narrowed portion at the distal end of the ampoule retainer (140) to assist in breaking the ampoule safely by applying isometric pressure to the ampoule neck. At the same end of the main body (110) as the ampoule neck lock (130) is a needle ledge guide (160) configured to protect the fingers of an operator during controlled breakage of the ampoule and to assist in withdrawing safely the contents of the ampoule into the syringe by guiding the syringe as the needle or the syringe tip enters the ampoule and by supporting the distal end of the syringe as the contents of the ampoule are withdrawn into the syringe.

In the embodiment shown in FIGS. 4-5, the ampoule lock (170) comprises a pin (173) fittable into a hole (177) in the side of the main body (110), at least one side of the pin (173) having pin teeth (175) and at least one side of the hole (177) having side teeth (176), with the side(s) of the pin (173) with pin teeth (175) matable with side(s) of the hole (177) having side teeth (176) so that the mating teeth (176, 177) form a one-way latch, preventing the pin (173) from being withdrawn after having been pushed in. Any other conventional means of providing a controllable, variable-length one-way latch can be used, for non-limiting example, a snap-fit type mechanism, with a plurality of snap-fit male portions matable with at least one snap-fit female portion or a plurality of snap-fit female portions matable with at least one snap-fit male portion.

The main body (110) preferably comprises a pen hanger (180) to enable the ampoule mate to be clipped to a pocket or belt, enabling medical personnel to carry the filled syringe (and attached ampoule) safely, conformably and conveniently. In the embodiment shown an FIGS. 4-5, the pen hanger (180) comprises, at its distal end, a tapping extension (185) that fits into a tapping hole (187) on the side of the main body (110). The tapping hole (187) extends into the bore of the ampoule holder (140), The pen hanger (180) with tapping extension (185) is configured to enable a user to tap the side of the ampoule in order to induce medicament in the neck or head of the ampoule to leave the neck or head and enter the body of the ampoule, thereby ensuring that substantially all of the medication in the ampoule is in the main body and can be drawn into the syringe. In use, a user would pull the distal portion of the pen hanger (180) outward, partially withdrawing the tapping extension (185) from the tapping hole (187). The pen hanger (180) material is sufficiently elastic so that, when the distal portion of the pen hanger (180) is released, the tapping extension (185) returns to its unextended configuration, rapping the side of the ampoule and inducing medicament in the neck or head of the ampoule to descend into the main body of the ampoule. The rapping process can be repeated as many times as desired.

The ampoule mate (1000) of FIGS. 4-5 comprises a lens holder (115) configured to hold a lens (not shown) to enlarge the text on the ampoule and make it easier to read. The lens can comprise a convex lens, a biconvex lens, a Fresnel lens, or any conventional system configured to readably enlarge text. The lens can be integral with the ampoule mate (1000), or can be attached to the main body (110) by a conventional means, such as, but not limited to, adhesive, a snap-fit, or teeth any combination of attachment means can be used, as long as the lens is not removable from the side of the main body (110) and can provide a readable enlarged view of information provided on a side of the ampoule.

Preferably, once the ampoule lock (170) has been locked, the ampoule is held firmly but rotatably in the ampoule mate (100, 1000) so that the ampoule can be rotated around its longitudinal axis while securely retained in the ampoule holder (150), thereby enabling the ampoule to rotated until the information on the side of the ampoule is visible through the lens.

Preferably, the end face (173) of the pin or arm (173) of the ampoule lock (170) is curved, to help ensure rotatability of the ampoule. In some embodiments, the diameter of the curvature matches that of the ampoule holder (150). In some embodiments, the curvature has a diameter approximately equal to that of the largest ampule fittable in the ampoule holder (150) or no less than that of the largest ampule fittable in the ampoule holder (150). For non-limiting example, if the largest ampule usable in the ampule mate is 2 cc, the diameter will be approximately 10.8 mm (10.75+0.5 mm tolerance), or will be no less than 10.8 mm.

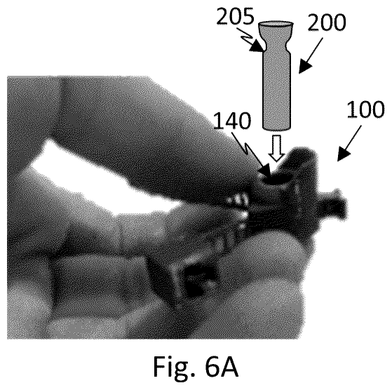

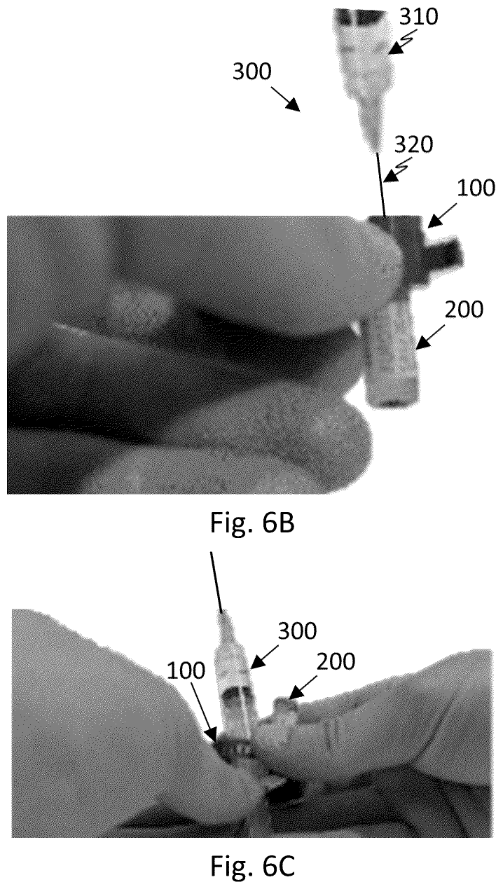

FIGS. 6A-C illustrate a method of use of the ampoule mate.

In a first step (FIG. 6A), the ampoule (200, shown schematically) is inserted fully into the ampoule holder (150) and the ampoule retainer (140) of the ampoule mate (100), so that the ampoule end (205) of the neck of the ampoule (200) rests against the ampoule neck lock (130). The ampoule lock (170) is then locked, fixing the ampoule (200) in position in the ampoule holder (150), with at least a portion of the neck of the ampoule (200) resting against the ampoule neck lock (130). For non-limiting example, the arm or pin (173) of the ampoule lock (170) is pressed inward until the ampoule (200) is held firmly in the ampoule holder (150) by the ampoule lock (170).

In a second step, the ampoule mate (100) and ampoule (200) are held in one hand and the head of the ampoule (200) (above the neck) is pressed toward the needle ledge guide (160). Since the neck of the ampoule (200) rests against the ampoule neck lock (130), an even pressure is exerted on at least a portion of the neck of the ampoule (200), facilitating safe breaking of the ampoule (200) neck, as well as a smooth rim to the broken neck.

In a third step (FIG. 6B), the needle (320) of the syringe (300) is inserted into the medicament ampoule (200) and the contents of the medicament ampoule (200) are drawn into the body (310) of the syringe (300).

The syringe (300) can now be attached to the ampoule mate (100) by the syringe lock (120).

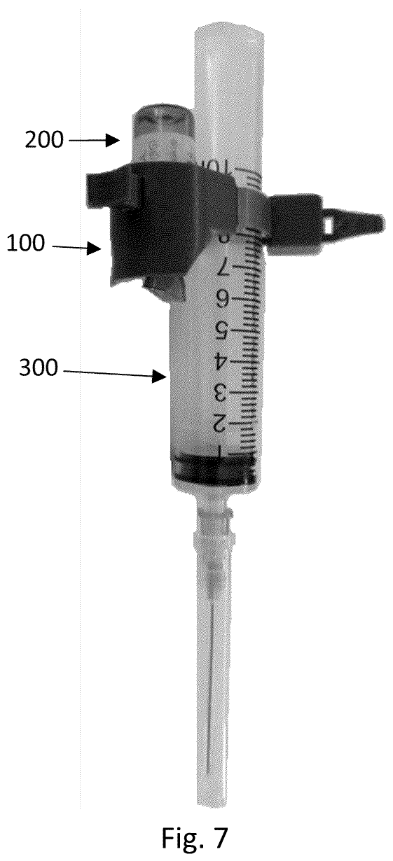

FIGS. 6C and 7 illustrate the fully-assembled system, ready for use. The syringe (300) and the ampoule (200) are held to the ampoule mate (100) firmly and in a difficult-to-remove manner. The system is easy and convenient to assemble and, fully assembled, it forms a compact package that is easy to store and convenient to use.

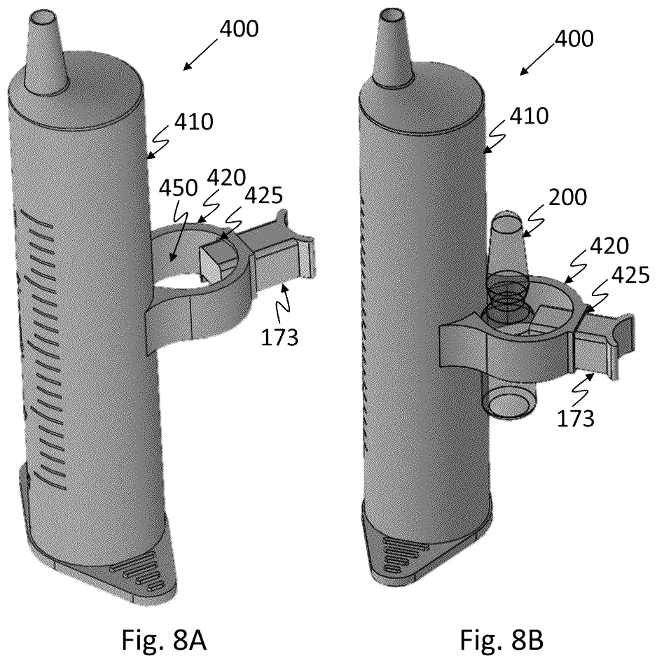

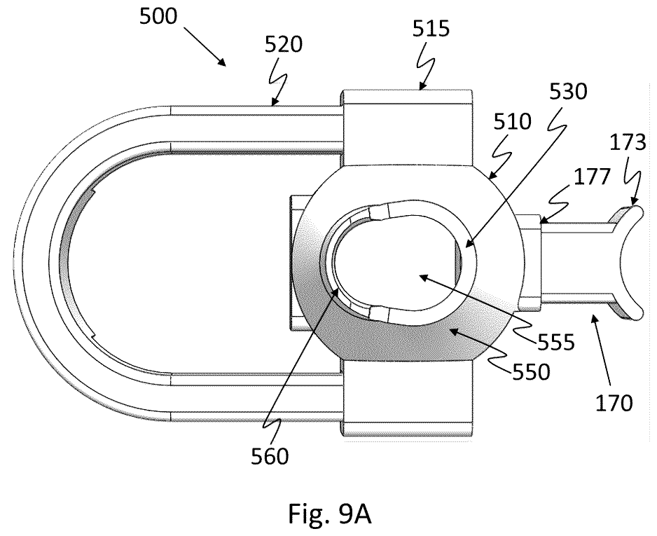

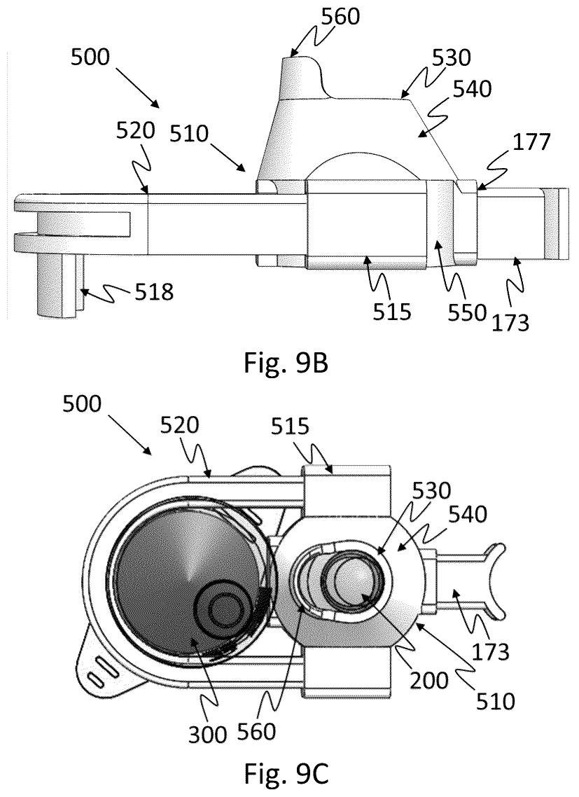

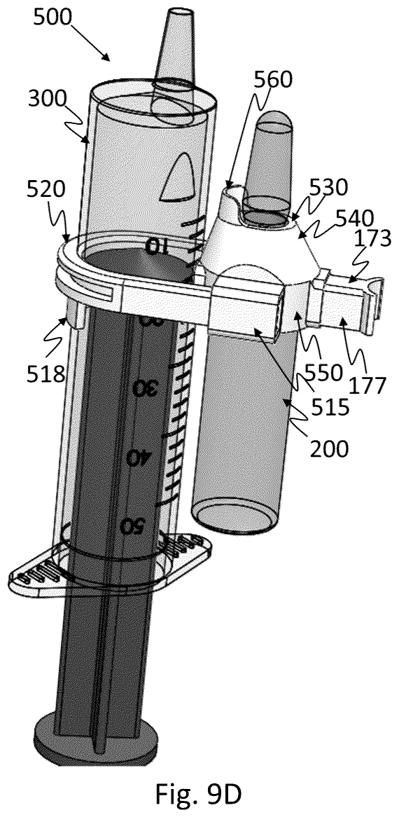

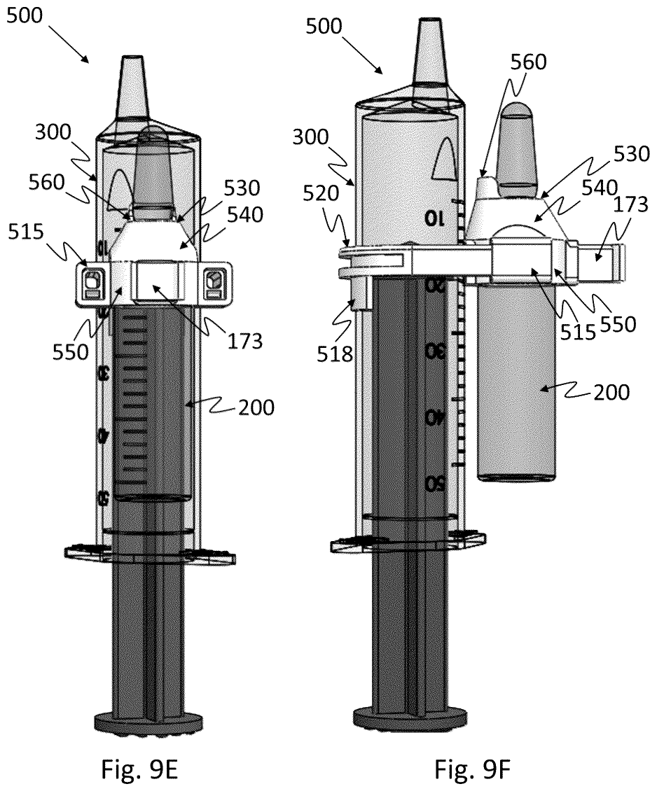

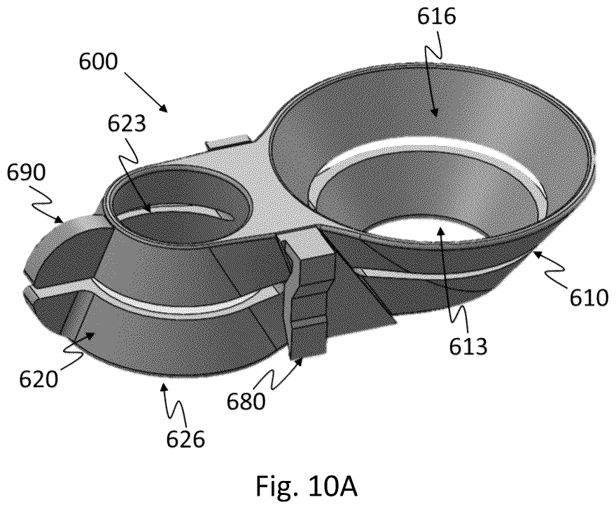

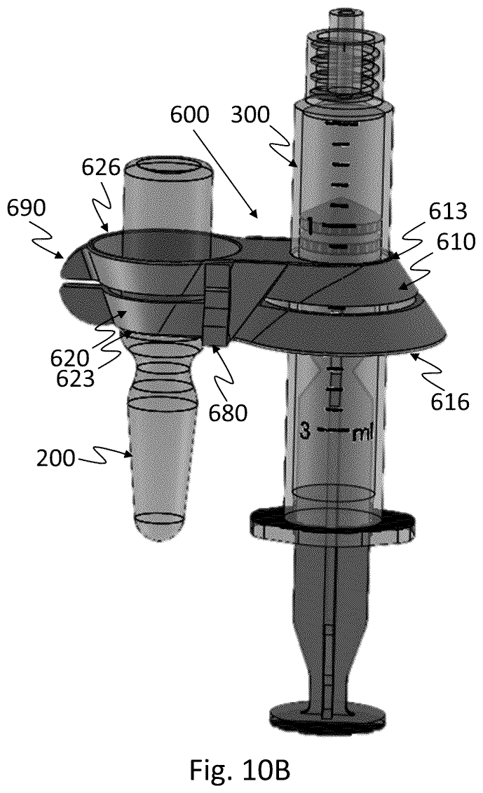

FIGS. 8A-B illustrate another embodiment of the ampoule mate (400) of the present invention, comprising a syringe (410) with an integral ampoule holder (420). FIG. 8A shows the ampoule mate (400) without an ampoule (200) and FIG. 8B shows the ampoule mate (400) holding an ampoule (200). In this embodiment, the ampoule holder (420), which is permanently attached to the side of the syringe (410), has an ampoule lock (173) to retain the ampoule (200). The ampoule holder (420) has a substantially cylindrical bore (455) sized and shaped to accept ampoules up to 10 cc. The ampoule lock (173) fits into a hole (425) in the side of the ampoule holder (420). The ampoule holder (420), ampoule lock (173) or both comprise a non-return mechanism so that the ampoule lock (173) can not be withdrawn from the ampoule holder (420) and, if pushed inward, can not be moved outward, thus securely holding the ampoule in the ampoule holder (420). Any conventional non-return mechanism can be used. For non-limiting example, the non-return mechanism can be a ratchet with teeth (not shown, see FIGS. 4B and 5B) on at least one side of the ampoule lock (473) mating with teeth on the same side(s) of the hole (425) or a snap-fit type mechanism, with a plurality of snap-fit male portions matable with at least one snap-fit female portion or a plurality of snap-fit female portions matable with at least one snap-fit male portion. In this embodiment (400), the ampoule mate does not comprise a surface or edge to break the neck of an ampoule. The ampoule will be opened as appropriate for that type of ampoule and the medicament withdrawn into the syringe before insertion of the ampoule into the ampoule holder.

An advantage of this type of embodiment is that it has fewer parts; there is no need for any attachment means to hold the ampoule holder to the syringe.

The embodiment (400) as shown FIGS. 8A-B does not have a pen hanger (180, see FIGS. 4-5). Other variants of this embodiment comprise a pen hanger (180, see FIGS. 4-5), either attached to the syringe or attached to the ampoule holder (420). The pen hanger (180, see FIGS. 4-5) enables the ampoule mate to be clipped to a pocket or belt, enabling medical personnel to carry the filled syringe (and attached ampoule) safely, conformably and conveniently.

FIGS. 9A-F schematically illustrate another embodiment of an ampoule mate (500). FIGS. 9A-B illustrate the ampoule mate (500) without an attached ampoule (200) and FIGS. 9C-F illustrate the ampoule mate (500) with an attached ampoule (200). FIG. 9A shows a top view and FIG. 9B shows a side view of the ampoule mate (500) without an attached ampoule (200). FIG. 9C shows a top view, FIG. 9D shows a perspective view and FIGS. 9E-F show side view of the ampoule mate (500) with an attached ampoule (200).

In this embodiment, the ampoule mate (500) comprises a body (510) comprising an ampoule holder (550) with an ampoule retainer (540) at one end of the ampoule holder (550). The ampoule holder (550) and ampoule retainer (540) form a throughgoing bore (555) along the longitudinal axis of the main body. The bore (555) terminates in an ampoule neck lock (530), a narrowed portion at the distal end of the ampoule retainer (540) to assist in breaking the ampoule safely by applying isometric pressure to the ampoule neck. At the same end of the main body (510) as the ampoule neck lock (530) is a needle ledge guide (560) configured to protect the fingers of an operator during controlled breakage of the ampoule and to assist in withdrawing safely the contents of the ampoule into the syringe by guiding the syringe as the needle or the syringe tip enters the ampoule and by supporting the distal end of the syringe as the contents of the ampoule are withdrawn into the syringe.

The ampoule lock (170) comprises a pin (173) fittable into a hole (177) in the side of the main body (510). Any conventional means of providing a controllable, variable-length one-way latch can be used, for non-limiting example, at least one mating set comprising teeth on a side of the pin and teeth on the same side of the hole (177), a snap-fit type mechanism, with a plurality of snap-fit male portions matable with at least one snap-fit female portion or a plurality of snap-fit female portions matable with at least one snap-fit male portion.

The main body (510) preferably comprises a pen hanger (180, see FIGS. 4-5) to enable the ampoule mate to be clipped to a pocket or belt, enabling medical personnel to carry the filled syringe (and attached ampoule) safely, conformably and conveniently.

In the embodiment of FIG. 9, the syringe lock comprises a U-shaped portion (520) and a gripping portion (515) configured to retain a syringe (300) securely. The U-shaped portion (520) is configured to hold the syringe (300) against the side of the main body (510), on the opposite side of the main body (510) from the syringe lock (170). The ends of the U-shaped portion (520) and the gripping portion (515) comprise a one-way latch. Any conventional means of providing a controllable, variable-length one-way latch can be used. Each end of the U-shaped portion (520) can comprise, for non-limiting example, at least one mating set comprising teeth on a side of the end of the U and teeth on the same side of the hole in the gripping portion (515), a snap-fit type mechanism, with a plurality of snap-fit male portions matable with at least one snap-fit female portion or a plurality of snap-fit female portions matable with at least one snap-fit male portion.

FIG. 10A-B schematically illustrates another embodiment of the ampoule mate (600), where the syringe holder (620) and the ampoule holder (610) each comprise a frustum of a cone. FIG. 10A shows the ampoule mate (600) alone, while FIG. 10B shows the ampoule mate (600) holding a syringe (300) and ampoule (200).

In the embodiment shown, the cones comprise two parts, a wider proximal end (616, 626) and a narrower distal end (613, 623). The syringe (300) is inserted into the wider proximal end (616) of the syringe holder (610), which guides it into the narrower distal end (613), thereby holding a syringe (300) securely. Similarly, the ampoule (200) is inserted into the wider proximal end (626) of the ampoule holder (620), which guides it into the narrower distal end (623), which holds the ampoule (200) securely. The flanges (690)on the upper and lower portions of the ampoule holder (620), when pressed together, ease the process of inserting the ampoule (200) into the ampoule holder (620). In some embodiments, there are similar flanges (not shown) on the syringe holder (610).

In the embodiment shown, the wider proximal end (616, 626) face in opposite directions so that the longitudinal axes of the ampoule (200) and the syringe (300) are substantially parallel. The material of at least a portion of the cones is flexible so that, upon insertion of a syringe (300) into the large frustum (620) or insertion of an ampoule (200) into the small frustum (610), the cone material stretches so as to hold the syringe (300) or ampoule (200) securely.

The embodiment shown comprises a pen hanger (680) to enable the ampoule mate to be clipped to a pocket or belt, enabling medical personnel to carry the filled syringe (and attached ampoule) safely, conformably and conveniently.

Any embodiment of the ampoule mate can comprise a pen hanger, a magnifying lens, an ampoule neck lock, a needle ledge guide (160) or any combination thereof.

17

1.-21. (canceled)

22. An ampoule mate (100, 1000) for retaining a medicament ampoule (200) in communication with a syringe, comprising:
a main body (110) with a throughgoing bore, said bore comprising a cylindrical ampoule holder (150) in a proximal portion of said bore and a narrowing ampoule retainer (140) with circular cross-section in a distal portion of said bore, the bore (150) being parallel to a main longitudinal axis of the main body (110);
wherein the main body further comprises an ampoule neck lock (130) at a distal end of the bore configured to assist in ensuring, in use, that a neck of the medicament ampoule (200) breaks evenly when a sidewards pressure is applied to the neck of the medicament ampoule (200);
a syringe lock (120) comprising two arms extending from a side (112) of the main body (110), the syringe lock (120) configured to firmly hold the syringe between the arms;
wherein the side (112) of the main body (110) between the arms of the syringe lock (120) having a concave curve, the concave curve having a longitudinal axis parallel to the main longitudinal axis of the main body (110); and
an ampoule lock (170) extending from a side of the main body (110) outside the syringe lock (120) to said cylindrical ampoule holder (150), the ampoule lock (170) configured to firmly hold the medicament ampoule (200).

23. The ampoule mate of claim 22, wherein said ampoule lock (170) is configured to accept an ampoule of a size between 1 cc and 10 cc.

24. The ampoule mate of claim 22, wherein said syringe lock (120) comprises a cable tie.

25. The ampoule mate of claim 22, wherein said main body (110) further comprises a pen hanger (180).

26. The ampoule mate of claim 25, wherein a distal end of said pen hanger (180) comprises a tapping extension (185).

27. The ampoule mate of claim 22, wherein said ampoule lock (170) comprises a male member (173) selected from a group consisting of a pin or an arm, said male member configured to securely hold said ampoule (200) in said ampoule mate (100, 1000), said male member snugly and fittable into a hole (177) in a side of said main body (110) outside said syringe lock (120), said hole (177) extending from an outside of said main body (110) to said ampoule holder (150) bore, a distal end of said male member (173) extendable into said ampoule holder (150) bore, said male member (173) configured to be movable inwards towards said ampoule holder (150) bore (150) but not movable outwards from said bore.

28. The ampoule mate of claim 27, wherein said ampoule (200), when held snugly within said ampoule holder (150) by said male member (173), is rotatable about an ampoule (200) main longitudinal axis.

29. The ampoule mate of claim 22, additionally comprising a needle ledge guide (160) at a distal end of said main body (110) for protecting the fingers of the operator during controlled breakage of said ampoule.

30. The ampoule mate of claim 22, additionally comprising a magnifying lens on a side of said main body (110) outside of said syringe lock (120).

31. A method of preparing a syringe for use, comprising steps of:
providing the syringe (300) with a body (310) and a needle (320);
providing a medicament ampoule (200) of medication;
providing an ampoule mate (100, 1000) comprising:
a main body (110) with a throughgoing bore, said bore comprising a cylindrical ampoule holder (150) in a proximal portion of said bore and a narrowing ampoule retainer (140) with circular cross-section in a distal portion of said bore, the bore being parallel to a main longitudinal axis of the main body (110);
wherein the main body further comprises an ampoule neck lock (130) at a distal end of the bore configured to assist in ensuring, in use, that a neck of the medicament ampoule (200) breaks evenly when a sidewards pressure is applied to the neck of the medicament ampoule (200);
a syringe lock (120) comprising two arms extending from a side (112) of the main body (110), the syringe lock (120) configured to firmly hold the syringe between the arms;
wherein the side (112) of the main body between the arms of the syringe lock (120) having a concave curve, the concave curve having a longitudinal axis parallel to the main longitudinal axis of the main body (110); and
an ampoule lock (170) extending from a side of the main body (110) outside the syringe lock (120) to said cylindrical ampoule holder (150), the ampoule lock (170) configured to firmly hold the medicament ampoule; (200).
inserting the medicament ampoule (200) into the ampoule holder (150) and the ampoule retainer (140) of the ampoule mate (100, 1000);
pushing the neck of the medicament ampoule (200) against an ampoule neck lock (130) at a distal end of the ampoule retainer (140) and breaking the neck of the medicament ampoule (200);
inserting the needle (320) into the ampoule (200) and drawing the contents of the medicament ampoule (200) into the syringe (300);
placing the side of the syringe body (310) against a concavely curved portion (112) of the side of the ampoule mate (100, 1000); and
closing the syringe lock (120), thereby attaching the syringe (300) to the ampoule mate (100).

32. The method of claim 31, additionally comprising a step of configuring said ampoule lock (170) to accept an ampoule of a size between 1 cc and 10 cc.

33. The method of claim 31, additionally comprising a step of said syringe lock (120) comprising a cable tie.

34. The method of claim 31, additionally comprising a step of providing said main body (110) comprising a pen hanger (180).

35. The method of claim 34, additionally comprising a step of providing a distal end of said pen hanger (180) comprising a tapping extension (185).

36. The method of claim 35, additionally comprising a step of inducing said tapping extension (185) to tap a side of said medicament ampoule (200).

37. The method of claim 31, additionally comprising a step of providing said ampoule lock (170) comprising a male member (173) selected from a group consisting of a pin or an arm, said male member (173) configured to securely hold said medicament ampoule (200) (200) in said ampoule mate (100, 1000), said male member snugly fittable into a hole (177) in a side of said main body (110) outside said syringe lock (120), said hole (177) extending from an outside of said main body (110) to said bore (150), an end of said pin (173) extending into said ampoule holder (150) bore, said male member (173) configured to be movable inwards towards said ampoule holder (150) bore but not movable outwards from said ampoule holder (150) bore.

38. (canceled)

39. The method of claim 31, additionally comprising a step of providing said main body (110) with a needle ledge guide (160) at a distal end of said main body (110) for protecting the fingers of the operator during controlled breakage of said ampoule.

40. The method of claim 31, additionally comprising a step of providing a magnifying lens on a side of said main body (110) outside of said syringe lock (120).
